SEQUENCE LISTING (2 sequences)

SEQ ID NO: 1 - protein, 58 aa, Artificial Sequence
  Description of Artificial sequence Synthetic Donor Peptide
CGGGGXXXXXXGSLAVVLQRRDWENPGVTQLNRLAAHPPFASWRNSEEARTDCPSQQL

SEQ ID NO: 2 - protein, 63 aa, Artificial Sequence
  Description of Artificial sequence Synthetic Donor Peptide
CGGGGRVDEPFSPGEKGSLAVVLQRRDWENPGVTQLNRLAAHPPFASWRNSEEARTDCPSQQL

(19) United States
(12) Patent Application Publication
(10) Pub. No.: US 2016/0068902 A1
(43) Pub. Date: Mar. 10, 2016

(54) SEQUENCING NUCLEIC ACIDS BY ENZYME ACTIVATION

(71) Applicant: CAERUS MOLECULAR DIAGNOSTICS, Mountain View, CA (US)

(72) Inventor: Javier Farinas, Los Altos, CA (US)

(21) Appl. No.: 14/759,892

(22) PCT Filed: Jan. 9, 2014
(86) PCT No.: PCT/US14/10922

§ 371 Date: Jul. 8, 2015

Related U.S. Application Data
(60) Provisional application No. 61/750,453, filed on Jan. 9, 2013.
(60) Provisional application No. 61/894,399, filed on Oct. 22, 2013.

(51) Int. Cl. C12Q 1/68; C07H 19/207; C07H 19/10; B01J 19/00

(52) U.S. Cl. CPC C12Q 1/6874; B01J 19/0046; C07H 19/207; C07H 19/10; B01J 2219/00596; B01J 2219/00722; B01J 2219/00585

(57) ABSTRACT

Provided herein are methods of nucleic acid sequencing using nucleotides with labels attached to the phosphate group so that incorporation of such nucleotides into a primed template results in formation of a phospho-label. Treatment of the phospho-label with a phophatase generates a free label which can be detected in a variety of ways. The labels can include, e.g., chemiluminescent labels, chemiluminescent substrates and enzyme activators. Also provided are reagents such as nucleotides phospholinked to labels such as enzyme activators.

### CROSS-REFERENCE TO RELATED APPLICATIONS

This application claims priority to and benefit of U.S. Provisional Application Ser. No. 61/750,453, filed Jan. 9, 2013, and U.S. Provisional Application Ser. No. 61/894,399, filed Oct. 22, 2013, the disclosures of which are incorporated herein by reference in their entirety for all purposes.

### STATEMENT REGARDING FEDERALLY SPONSORED RESEARCH

This invention was made in part with support of the National Institutes of Health through grants 1R43HG005865-01 and 1R21 HG006030-01. The government has certain rights in the invention.

### FIELD OF THE INVENTION

The present disclosure is in the field of nucleic acid sequencing. In particular, described herein are methods for quickly sequencing nucleic acids.

### BACKGROUND OF THE INVENTION

Nucleic acid sequencing is an important part of medical research, diagnostics, industrial processing, crop and animal breeding, and many other fields. For example, sequencing is used to diagnose disease conditions, detect infectious organisms, identify individuals in forensic applications and discover disease-causing genes.

A commonly used method of nucleic acid sequencing is Sanger sequencing.¹ The Sanger method uses dideoxynucleotide triphosphates (ddNTPs) as DNA chain terminators to generate a set of nucleic acid fragments which differ in length by one nucleotide. The dideoxynucleotides (e.g. ddATP, ddGTP, ddCTP and ddTTP) which cause chain termination can be identified by labeling each dideoxynucleotide with a distinguishable detectable label.² The labeled DNA fragments are size separated by gel electrophoresis with single nucleotide resolution. Electrophoretic separation is performed in slab gels, capillaries or microfluidic devices using denaturing polyacrylamide-urea gels or other sieving polymer matrices. The DNA sequence is defined by the order in which the dideoxynucleotide terminated fragments appear. One of the drawbacks of Sanger sequencing is the large amount of sample preparation required to sequence nucleic acids which results in high cost.

Recently, new methods have been developed for ultra high throughput sequencing of nucleic acids based on highly parallel schemes which greatly reduce the per-base cost of sequencing. For example, one such method uses terminal phosphate—labeled fluorogenic nucleotides (TPLFN) in resealable polydimethylsiloxane (PDMS) microreactors¹⁴. In the presence of phosphatase, primer extension by DNA polymerase using nonfluorescent TPLFNs generates fluorophores, which are confined in the microreactors and detected. Most of these new methods use an in vitro cloning step to generate many spatially localized copies of individual template nucleic acid molecules in a sample. For example, one method for generating a library of clonally amplified template molecules is emulsion PCR.³ A water-in-oil emulsion is formed such that the aqueous droplets dispersed in the oil phase contain amplification reagents such as polymerase chain reaction (PCR) reagents and limiting amounts of primer-coated beads and templates. The beads and templates are added in amounts such that most beads bind zero or one template. Additionally, most droplets have zero or one bead. A PCR reaction is carried out to amplify the templates. Usually, the amplicons are bound to the beads by primers covalently attached to the beads. After breaking the emulsion, the beads can be processed in parallel either in a sequencing-by-synthesis or ligation method to obtain sequence information.⁴ Another method for in vitro clonal amplification is “bridge PCR”, where fragments are amplified using primers attached to a solid surface.⁵ Both of these methods produce many physically isolated locations which each contains many copies of a single template.

Once clonal amplification is completed, various methods can be used to read out a sequence. The 454 method (Roche, Branford, Conn.) uses a fiber optic slide with millions of individual wells for highly parallel pyrosequencing reactions.⁴ Another method involves incorporation of fluorescently labeled reversible terminator nucleotides into clonal amplicons distributed on the surface of a flow cell.⁶ Yet another method uses emulsion PCR to generate clonally amplified libraries which are deposited on a glass slide. A series of probe oligos are ligated to the bead-bound nucleic acids to read out the sequence.⁷

IIlumina (San Diego, Calif.) and Ion Torrent (Life Technologies, Carlsbad, Calif.) have developed very high throughput and low cost sequencers¹⁵. Limits on detection signal-to-noise ratios have required that each system generate hundreds to hundreds of thousands of clonal template copies in order to achieve low error rates. However, the use of multiple template copies per clone leads to dephasing due to incomplete incorporation per dNTP addition cycle which limits read lengths to about 500 nucleotides¹⁶. Current single molecule sequencing methods such as the SMART technology (Pacific Biosciences, Menlo Park, Calif.) or nanopore methods result in read lengths of tens of thousands of bases by getting rid of the dephasing limitation¹⁷. However, such methods have very high raw error rates (4-15%). Long reads enable simpler assembly of complex genomes and allow for accurate phasing of SNPs without the need for complex and expensive sample preparation procedures¹⁸. In addition, eliminating the need for template amplification speeds up and lowers the cost of sample preparation while reducing the occurrence of amplification bias and errors.

Previous work on the use of terminal phosphate labeled dNTPs by Pacific Biosciences, GE LifeSciences, and the Xie group have generated a single reporter molecule from each incorporated dNTP molecule¹⁹. This has led to either high noise, the need for thousands of template copies or complex detection schemes.

The integrated solid state sensor developed by Ion Torrent allows for low cost instrumentation by eliminating the need for expensive, sensitive optical components²⁰. Such sensors benefit from the investment in semiconductor manufacturing infrastructure made by the computer industry. This allows the feature size and sensor manufacturing costs to decrease by piggybacking on the semiconductor industry's move to smaller and smaller wavelength photolithographic manufacturing processes. However, the Ion Torrent integrated CMOS pH sensor requires the generation of many protons to achieve adequate sensitivity.

Despite the wide usage of Sanger sequencing and the host of newly developed high throughput sequencing methods, certain deficiencies persist in current nucleic acid sequencing methods. Some of the drawbacks of Sanger sequencing are the large amount of sample preparation required to sequence nucleic acids and the high per-base cost of sequencing. New high throughput sequencing methods can analyze millions or billions of clones in parallel but are often limited to obtaining 200-500 nucleotides of sequence information per clone. For some applications however, it is important to obtain thousands of nucleotides of information per clone. For example, in de novo genome sequencing, long read lengths are required to close gaps.⁸ In another example, long read lengths are required to unambiguously detect linked mutations in HIV genotyping or HLA allelotyping applications.⁹

While the cost per base of next generation technologies is much lower than for traditional Sanger sequencing, there is still a need for methods which have a lower cost than Sanger or next generation methods or which have long read lengths and low error rates.

The present invention overcomes these and other problems in the art. A more complete understanding of the invention will be obtained upon complete review of the following.

### SUMMARY OF THE INVENTION

The present invention provides reagents, methods, and systems for sequencing nucleic acids using detection of a phospho-product of nucleic acid incorporation during nucleic acid polymerization. In various embodiments herein, the methods use an enzyme to convert a product produced from a sequencing reaction into many copies of a readily detectable reporter molecule. More specifically, in many embodiments sequencing is performed using nucleotides that are labeled at the terminal phosphate. The label can be an enzyme activator or can be converted to an enzyme activator. For example, sequencing can take place in a sequencing-by-synthesis scheme. Upon incorporation of a nucleotide onto a primed nucleic acid template (for example, either DNA or RNA), an activator is released which can increase the activity of an activatable enzyme or a species is released which can be converted into an activator which can increase the activity of an activatable enzyme. The activator uncoupled from the nucleotide activates the enzyme more than the activator does when it is still coupled to the nucleotide. Thus, each activated enzyme can rapidly generate a multitude of detectable products thereby amplifying the detectable signal from the original nucleotide incorporation. The generation of multiple copies of a reporter makes it easier to detect nucleotide incorporation. Detection of the reporter indicates nucleotide incorporation which is used to sequence the primed nucleic acid template. This can improve the overall signal-to-noise ratio of the system. The ratio can be high enough to allow single molecule sequencing with low noise. Such single molecule sequencing simplifies sample preparation and enables very long read lengths by eliminating dephasing limitations.

The methods of the invention can be used for sequencing a primed target nucleic acid. To sequence a nucleic acid, one or more clonal copies of a target nucleic acid can be spatially separated from other nucleic acid templates. To the nucleic acid, a reaction mixture containing a polymerase activity and at least one species of nucleotide labeled at the phosphate such that the label substantially does not activate an enzyme until after incorporation of said nucleotide into the nucleic acid by the polymerase. After release of the phospho-label, the label acts as an enzyme activator or can be converted to an enzyme activator. After allowing for the template-dependent addition of the nucleotide to the one or more copies of the target nucleic acid, the sequence of the target nucleic acid can be determined by detecting incorporation of said nucleotide during template-dependent polymerization by detecting an activatable enzyme activity resulting from release of the phospho-label.

Spatial separation can be achieved in a variety of ways. For example, different target nucleic acids can be placed in microreactors. More than one microreactor can be used. In some embodiments, the microreactors can be in fluidic contact with one another. For example, arrays of more than 10, more than 100, more than 1,000, more than 100,000, more than 10,000,000 or more than 1,000,000,000 microreactors can be used. The microreactors can be open or reversibly sealable. For example, in some embodiments the microreactors can be an array of micron sized (0.1 to 100 microns) wells. For example, such wells can be made in substrates made from plastic, glass, quartz, silicon, metals etc. The microreactors can also be made from water-in-oil emulsions. For example, water droplets can be made such that the contents of each droplet can be kept separate from the contents of other droplets. The microreactors can also be made by separating water droplets on a surface. For example, arrays of hydrophilic patches on a hydrophobic substrate can be used to generate separate aqueous compartments. Separation can also be achieved by attaching nucleic acids to a surface so that nucleic acid clones are spatially separated.

In various embodiments the target nucleic acid clonal copies can be distributed to separate compartments so that most of the compartments contain clonal copies from no more than a single clone. The clonal copies can be placed in the compartments in a variety of ways. For example, clonal copies can be attached to a bead and the bead placed into the compartment. As another example, a rolony can be covalently or non-covalently (e.g. via a biotin-avidin linkage) attached to the surface of a compartment. As another example, a single copy of a nucleic acid can be placed in a compartment by attaching the nucleic acid to a bead and placing the bead in the compartment, by covalently attaching the nucleic acid to the surface of the compartment, by non-covalently attaching the nucleic acid to the surface of the compartment, or by binding a nucleic acid to a polymerase which is kept in the compartment either by covalent or non-covalent attachment to the surface of the compartment or by covalent or non-covalent attachment to a bead which is placed in the compartment.

The clonal copies in the various embodiments can contain different numbers of clonal copies of a target nucleic acid(s). For example, a single copy of a nucleic acid can be used. In other cases, fewer than 10 copies, fewer than 100, fewer than 1,000, fewer than 10,000, fewer than 100,000 or fewer than 1,000,000 copies are used. In some embodiments, the phospho-label released by incorporation of the phospho-labeled nulecotide into the primed target nucleic acid activates an enzyme. In some embodiments, the reaction mixture also contains one or more conversion enzymes that converts the phospho-label, that itself is not an enzyme activator, into an enzyme activator capable of activating an enzyme. For example, the conversion enzyme can be alkaline phosphatase, acid phosphatase, neutral phosphates, galactosidase, horseradish peroxidase, phosphodiesterase, phosphotriesterase, pyruvate kinase, lactic dehydrogenase, maltose phosphorylase, glucose oxidase, lipase, beta amylase, proteases, kinases, or combinations thereof. In some embodiments, the label is attached to the terminal phosphate of a nucleotide. A phospho-label is cleaved from the nucleotide when the nucleotide is incorporated into the primed target nucleic acid template complex by a polymerase activity. Thus, in various embodiments, either the phospho-label is an enzyme activator or it can be converted to an enzyme activator. The phospho-label optionally can be converted to a label by the presence of a phosphatase enzyme. Once dephosphorylated, the label can activate an enzyme. For example, a maltose label can be coupled to a nucleotide. Upon incorporation of the nucleotide into a primed template, phospho-maltose is released. The phospho-maltose can be used to activate a phospho-maltose activated enzyme. Alternatively, phospho-maltose can be dephosphorylated (i.e. converted to maltose) to yield maltose which can then be used to activate a maltose activated enzyme. Alternatively, the phospho-maltose can be converted to glucose which can then be used to activate a glucose activated enzyme. Alternatively, the phospho-maltose can be dephosphorylated and then converted to glucose which can then activate a glucose activated enzyme. As will be clear to those skilled in the art, labels other than maltose can optionally be used. In all such cases, the activated enzyme can optionally generate a large number of reporters which can then detected. Detection of the generation of the reporters thus indicates the incorporation of the nucleotide. Incorporation of the nucleotide is used to sequence the target nucleic acid.

In the embodiments herein, sequencing can be performed by adding more than one labeled nucleotide. The labeled nucleotides optionally can be added at the same time or sequentially. The label on each nucleotide can be the same or can optionally be different so that each label activates a separate enzyme activity. For example, sequential addition of four labeled nucleotides can be conducted until the target nucleic acid is sequenced. The method can be used to obtain the sequence for more than 1, 10, 25, 100, 300, 1,000 or 10,000 bases of said target nucleic acid. In another example, each species of nucleotide can have a distinguishable label which once released is an enzyme activator or can be converted to an enzyme activator of a distinguishable enzyme activity.

The methods of the invention can optionally be performed with open, sealed or resealable microreactors or compartments. For example, a microwell array can be reversibly sealed by pressing a gasket (e.g. one made from PDMS) onto the top of wells. The gasket can then be lifted from the array to allow for exchange of reaction mixtures. For example, exchange of the components from a resealable microreactor can occur through unsealing the reactor, removing the mixture in solution phase, introducing a second mixture in solution phase, and resealing the microreactor. The microreactors can also be sealed with a water-immiscible liquid. Sealing lowers the rate at which reagents can move from inside to the outside of the compartments. In some embodiments, such sealing is not absolute so that a small rate of exchange is possible even in the presence of the seal. For example, the presence of the seal can reduce the diffusive transport of reagents out of a microreactor by more than 90%, more than 99% or more than 99.9% while still allowing an electrical current to be conducted from the compartment to the bulk fluid.

A variety of nucleic acid replicating catalysts can be used to incorporate the labeled nucleotide onto the primed nucleic acid template in the embodiments herein. For example, a DNA polymerase, RNA polymerase, ligase, reverse transcriptase, or RNA-dependent RNA polymerase can be used in various embodiments. The target nucleic acid template optionally can be primed with an oligonucleotide primer or can be self-primed. The nucleic acid template can be RNA, DNA or other nucleic acids capable of forming complimentary pairs.

The nucleotides used can also optionally comprise a reversible terminator. After incorporation of the nucleotide and detection of the activatable enzyme activity, the incorporated nucleotide can be reacted to release the termination moiety. Subsequent rounds of polymerization can then proceed. For example, sequencing can be performed by adding one or more species of nucleotide with each species having either the same or a different label. When separate labels are used, the labels can activate distinguishable enzymes, thus allowing determination of which nucleotide is being incorporated based on the particular activated enzyme activity. For example, each label can optionally activate a distinguishable enzyme such that the enzymes would each use fluorogenic substrates to generate separate fluorophores which are distinguished for example based on the emission spectra.

In some embodiments, it is desirable to trigger the start of the polymerization only once the microreactors have been sealed. For example, this can be done by using a temperature sensitive polymerase. For example, BST DNA polymerase can be used so that little nucleotide incorporation takes place when the microreactors are cooled to 15° C. or lower but incorporation takes place once the microreactors are heated to 30° C. or higher.

As an example, the methods herein can be used for single copy sequencing of a library of nucleic acid templates by a) immobilizing in individual microreactors a single target nucleic acid (e.g., optionally a primed target nucleic acid or a target nucleic acid along with one or more primers; b) cooling the microreactor to below 15° C.; c) introducing to the microreactor a reaction mixture comprising a nucleic acid replicating catalyst, and a single species of nucleotide comprising a first base and a first label that substantially does not activate an enzyme until after incorporation of said nucleotide into a nucleic acid based on complementarity to said target nucleic acid; d) sealing said microreactor and heating said microreactor to 30° C. or higher; e) allowing template-dependent replication of said target nucleic acid; f) sequencing said target nucleic acid by detecting incorporation of said nucleotide during or after template-dependent replication by detecting enzyme activity resulting from said first label either in a phosphorylated or dephosphorylated state or upon conversion into an enzyme activator; g) repeating steps b)-f) sequentially with the four species of nucleotides.

Sequencing a nucleic acid with the methods, etc. herein can be conducted in a system comprising a plurality of microreactors that are each capable of holding an immobilized single target nucleic acid or plurality of copies of said target nucleic acid, a mixture in solution phase of a nucleic acid replicating catalyst, and a single species of nucleotide that comprises a label that substantially does not activate an enzyme until after incorporation of said nucleotide into a nucleic acid based on complementarity to said target nucleic acid; a fluorescence or luminescence microscope or a luminescence or pH CMOS sensor for detecting said plurality of microreactors to sequence target nucleic acids in said microreactors by detecting in each microreactor the incorporation of an individual nucleotide species during template-dependent replication of said single copy of said target nucleic acid by monitoring fluorescence, luminescence or pH from said labels resulting from incorporation of said at least one nucleotide; and a fluidic delivery system capable of delivering liquids from each of one or more (e.g., four) reservoirs to each of said plurality of microreactors.

The present invention provides novel methods and systems, as well as devices, reagents and reaction mixtures used in such methods and systems. In at least one aspect the invention comprises methods for sequencing a nucleic acid by: immobilizing a target nucleic acid or a plurality of target nucleic acids in a microreactor (which target nucleic acid or plurality of target nucleic acids can comprise one or more primers such as nucleic acid primers either in solution with or bound to or associated with one or more areas of the target nucleic acid(s)); introducing a mixture in solution phase to the microreactor comprising a nucleic acid replicating catalyst (e.g., a DNA polymerase, an RNA polymerase, a ligase, a reverse transcriptase, or an RNA-dependent RNA polymerase, etc.) or optionally a replicating catalyst bound to a microreactor and a first labeled nucleotide that has a first base and a first label where the label does not substantially activate a first activatable enzyme until after incorporation of the labeled nucleotide into a complementary nucleic acid that is complementary to the target nucleic acid (i.e., a nucleic acid that is replicated from the target nucleic acid) and optionally other nucleic acid replication buffers, non-labeled nucleotides, enzymes, etc.; performing template-dependent replication of the target nucleic acid or the members of the plurality of target nucleic acids; and detecting incorporation of the first labeled nucleotide during template-dependent replication by monitoring the activatable enzyme activity resulting from interaction of the first phospho-label or a conversion product of the first phospho-label with the first enzyme after release of the first label from the first labeled nucleotide, thereby sequencing the target nucleic acid. In some embodiments, the methods comprise one or more conversion enzyme in the mixture solution (e.g., a conversion enzyme that can make the first label capable of activating the first enzyme). Examples of such conversion enzymes include, but are not limited to: an alkaline phosphatase, acid phosphatase, galactosidase, horseradish peroxidase, phosphodiesterase, phosphotriesterase, pyruvate kinase, lactic dehydrogenase, maltose phosphorylase, glucose oxidase, lipase, beta amylase, protease or any combination thereof of such enzymes. In some embodiments, the first label is attached to the terminal phosphate of the first labeled nucleotide and can be cleaved from the first labeled nucleotide during replication of the complementary nucleic acid (i.e., the replicated nucleic acid that is complementary to the target nucleic acid). In some embodiments, the steps of immobilizing a target nucleic acid (or a plurality of target nucleic acids) into a microreactor; introducing a mixture in solution phase that comprises a nucleic acid replicating catalyst and a labeled nucleotide (having a base and a label) to the microreactor; performing template-dependent replication of the target nucleic acid; and detecting incorporation of the labeled nucleotide into the replicating nucleic acid by monitoring enzyme activity that results from the label after the label is released from the labeled nucleotide once the nucleotide is incorporated into the replicating nucleic acid are repeated with (or can comprise) a second labeled nucleotide that comprises a second base and a second label which second label does not substantially activate a second enzyme until after the second labeled nucleotide is incorporated into the complementary nucleic acid that is replicated from the target nucleic acid. In some such embodiments, the first and second labels can be the same or different from each other and/or the first and second bases are different from each other and/or the first and second enzymes are the same or different from each. In yet other embodiments, the same steps can be repeated (or can comprise) a third labeled nucleotide that comprises a third base and a third label which label does not substantially activate a third enzyme until after incorporation of the third nucleotide into the complementary nucleic acid that is replicated from the target nucleic acid. In such embodiments, each of the first, second, and third labels can be the same or different from each other (or any two can be the same with the third being different), the first, second, and third bases are different from each other, and each of the first, second, and third enzymes can be the same or different from each other (or any two can be the same with the third being different). In still other embodiments, the same steps can be repeated (or can comprise) a fourth labeled nucleotide that comprises a fourth base and a fourth label which label does not substantially activate a fourth enzyme until after incorporation of the fourth nucleotide into the complementary nucleic acid that is replicated form the target nucleic acid. In such embodiments, each of the first, second, third, and fourth labels can be the same or different from each other (or any two or three can be the same with the other two or the fourth being different), the first, second, third, and fourth bases are different from each other, and each of the first, second, third, and fourth enzymes can be the same or different from each other (or any two or three can be the same with the other two or the fourth being different). In various embodiments, such steps can be sequentially repeated with the first, second, third, and/or fourth labeled nucleotides until the target nucleic acid is sequenced while in other embodiments, the steps can be repeated with the first, second, third, and/or fourth labeled nucleotides present concurrently until the target nucleic acid is sequenced. In the various embodiments, the microreactor can optionally be reversibly sealed. Also, in embodiments wherein the microreactor is reversibly sealed (e.g., with a water-immiscible liquid or a PDMS gasket), exchange of components from the microreactor when it is sealed can occur through unsealing the reactor, removing the mixture in solution phase, introducing an additional mixture in solution phase to the microreactor, and resealing the microreactor. In the various embodiments, the target nucleic acid can be DNA or RNA and the mixture in solution phase can comprise one or more nucleic acid primers (e.g., nucleic acid primers specific for one or more areas of the target nucleic acid(s)). In the various embodiments, the steps of the methods can be repeated to obtain the sequence for more than 1, more than 10, more than 25, more than 100, more than 300, more than 1,000 or more than 10,000 bases of the target nucleic acid. Also in the various embodiments, the target nucleic acid or plurality of target nucleic acids can be immobilized on one or more beads disposed in a microreactor and/or can be immobilized (e.g., via biotin) on one or more surfaces of a microreactor, and the target nucleic acids (e.g., the members of the plurality of target nucleic acids) can be produced by rolling circle amplification. In some embodiments, one or more of the first, second, third, or fourth labeled nucleotide can further comprise a reversible terminator, any of which can be optionally removed. Once incorporated and activatable enzyme activity is measured, the reversible terminator can be converted so that the nucleotide no longer prevents further polymerization. In some embodiments, the microreactor is cooled to 15° C. or lower prior to introducing the mixture in solution phase to the microreactor and/or the microreactor is heated to 30° C. or higher prior to or during performing template-dependent replication of the target nucleic acid. Some embodiments further comprise more than one target nucleic acid or more than one plurality of target nucleic acids where each target nucleic acid or each plurality of target nucleic acids is immobilized in one of a plurality of microreactors. In such embodiments, the steps of the methods (e.g., immobilizing, introducing, performing, and detecting) are performed for each target nucleic acid or each member of the plurality of target nucleic acids in the various microreactors. The plurality of microreactors can be super-Poisson loaded with the target nucleic acids or with the members of the pluralities of target nucleic acids.

In other aspects, the invention includes methods for sequencing a nucleic acid by immobilizing a target nucleic acid or a plurality of target nucleic acids in a microreactor; cooling the microreactor to 15° C. or lower; introducing a mixture in solution phase to the microreactor where the mixture comprises a nucleic acid replicating catalyst (e.g., e.g., a DNA polymerase, an RNA polymerase, a ligase, a reverse transcriptase, or an RNA-dependent RNA polymerase, etc.), and a first labeled nucleotide which labeled nucleotide comprises a first base and a first label which first label does not substantially activate a first enzyme until after incorporation of the nucleotide into a complementary nucleic acid that is complementary to the target nucleic acid, along with optionally other nucleic acid replication buffers, non-labeled nucleotides, enzymes, etc.; sealing the microreactor and heating it to 30° C. or higher; performing template-dependent replication of the target nucleic acid or of the members of the plurality of target nucleic acids; detecting incorporation of the nucleotide during or after template-dependent replication by monitoring enzyme activity resulting from interaction of the first label with the first enzyme after release of the first label from the first nucleotide, thereby sequencing the target nucleic acid; and repeating such steps with a second labeled nucleotide which second labeled nucleotide comprises a second base and a second label which second label does not substantially activate a second enzyme until after incorporation of the second nucleotide into the complementary nucleic acid; a third labeled nucleotide which comprises a third base and a third label which third label does not substantially activate a third enzyme until after incorporation of the third nucleotide into the complementary nucleic acid; and a fourth labeled nucleotide which comprises a fourth base and a fourth label which fourth label does not substantially activate a fourth enzyme until after incorporation of the fourth nucleotide into the complementary nucleic acid. In such embodiments, any of the first, second, third, and fourth labels can be the same or different (or any two or three can be the same with the other two or the fourth being different); the first, second, third, and fourth bases are different; and any of the first, second, third, and fourth enzymes can be the same or different (or any two or three can be the same with the other two or the fourth being different).

In some aspects, the invention comprises a system for sequencing a nucleic acid. Such systems can comprise a plurality of microreactors that are each capable of holding: an immobilized target nucleic acid or plurality of target nucleic acids, a mixture in solution phase of a nucleic acid replicating catalyst (e.g., a DNA polymerase, an RNA polymerase, a ligase, a reverse transcriptase, or an RNA-dependent RNA polymerase, etc.), and one or more labeled nucleotides which each comprises a label that does not substantially activate an enzyme until after incorporation of said nucleotide into a complementary nucleic acid that is complementarity to the target nucleic acid, and optionally other nucleic acid replication buffers, non-labeled nucleotides, enzymes, etc.; a fluorescence or luminescence microscope or a luminescence or pH or other CMOS sensor for monitoring the plurality of microreactors by detecting in each microreactor the incorporation of one or more labeled nucleotide into the complementary nucleic acid during or after template-dependent replication of the target nucleic acid by monitoring fluorescence, luminescence, pH or other detectable signal that results after cleaving of the labels from the labeled nucleotides and activation of an activatable enzyme which is used to generate a reporter when the nucleotides are incorporated into the complementary nucleic acid; and a fluidic delivery system that is capable of delivering liquids from one or more reservoirs to the members of the plurality of microreactors.

In the various embodiments of the aspects herein, the invention can comprise a single copy of a particular target nucleic acid in a microreactor, multiple copies (i.e, a plurality) of a particular target nucleic acid in a microreactor, or multiple copies of different target nucleic acids in a microreactor. Furthermore, in such various embodiments, with a plurality of microreactors there can optionally be either a single copy or a plurality of copies of either a particular or different nucleic acids in each microreactor as well as microreactors having either a single copy or a plurality of copies of either a particular or different nucleic acids along with non-target nucleic acids. Also in the various embodiments of the aspects herein, either the first labeled nucleotide, the second labeled nucleotide, the third labeled nucleotide, and the fourth labeled nucleotide can optionally be present in the microreactor(s) during the steps of the methods either at the same time (or in any subcombination of 1, 2, or 3 nucleotides) or added sequentially in any order. Additionally, in the various embodiments, the solutions or reagents/compounds present for the different steps in the methods can comprise appropriate buffers, reagents, substrates, etc. for the necessary nucleic acid replication activities (e.g., all necessary nucleotides, both labeled and/or unlabeled, etc.), conversion enzyme activities, and activator enzyme activities, etc.

In some aspects, the invention comprises compounds having an enzyme activator coupled to the terminal phosphate of a nucleotide. Examples of enzyme activators include, but are not limited to: maltose, glucose, histidine, camp, beta-galactosidase donor peptide or various ion channel ligands.

In other aspects, the invention comprises a compound comprising a nucleotide with a label attached to the terminal phosphate. The label or phospho-label can be an enzyme activator or can be converted to an enzyme activator. The nucleotide can contain 3, 4, 5 or more phosphates. In some embodiments, the nucleotides can have chemical groups which render the nucleotide a reversible terminator. In some aspects of the invention comprises compounds having the formula:

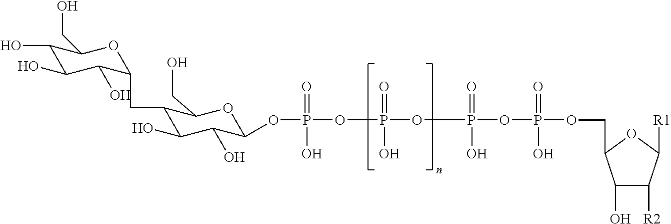

wherein n is 0 to 4, R1 is a nucleoside base and R2 is H, OH, or OMe.

The invention also includes kits, e.g., comprising a consumable of the invention. The kits can also include packaging materials, instructions for practicing the methods, control reagents and/or other reagents or components for amplification/sequencing of nucleic acids (e.g., control templates, probes, primers, nucleic acid amplification reagents, nucleotides (both unlabeled and/or labeled, etc.).

The methods, systems, devices, reagents/compounds, consumables, and kits of the invention can be used in any combination, e.g., with the kit providing consumables for use in a system or device of the invention, e.g., to practice the methods of the invention. Unless stated otherwise, steps of the methods optionally have corresponding structural features in the systems, devices, consumables or kits, and vice-versa.

### BRIEF DESCRIPTION OF THE DRAWINGS

FIG. 1 shows a schematic illustration of an embodiment of the invention with measurement of a phospho-product by enzyme activation. In this embodiment, the label is an activator. Release of the phospho-label is followed by conversion of the phospho-activator to the activator. The activator binds to an activatable enzyme thereby activating the enzyme. The activated enzyme is used to generate many copies of a detectable reporter product. The generation of the reporter is used to detect incorporation of the nucleotide and thus to sequence the target nucleic acid.

FIG. 2 shows a schematic illustration of an exemplary embodiment of the invention with measurement of a phospho-product by enzyme activation using the beta galactosidase system. Release of the phospho-product (i.e., the phospho-label) is followed by dephosphorylation and then proteolytic cleaved to generate the beta galactosidase donor peptide capable of activating the beta galactosidase acceptor enzyme.

FIG. 3 shows: 3(A) a schematic illustration of an exemplary embodiment of the invention with measurement of a phospho-product by enzyme activation using the maltose binding protein-lactamase/maltose system. The activator is maltose. FIG. 3(B) shows an example nucleotide labeled at the terminal phosphate with maltose which is the enzyme activator.

FIG. 4 illustrates an exemplary embodiment of the invention and shows: 4(A) Lactamase switch hydrolyses nitrocefin 210 times faster after maltose addition; 4(B) Kcat pH optimum; 4(C) Apparent nitrocefin Km; 4(D) Apparent maltose Km; and 4(E) Maltose triphosphate showing significant activation of the lactamase switch only upon dephosphorylation, thus demonstrating the ability of converting the phospho-maltose, which is not an enzyme activator, into maltose which is the enzyme activator.

FIG. 5 shows: 5(A) Fluorescence image of Si microreactor wells Scale bar 10 mm; and 5(B) Recovery after photobleaching of wells is very slow showing adequate well sealing. Thus, demonstrating the ability to reversibly seal microreactor compartments in various embodiments of the invention.

### DETAILED DESCRIPTION OF THE INVENTION

Before describing the present invention in detail, it is to be understood that this invention is not limited to particular devices, systems, or components, etc., which can, of course, vary. It is also to be understood that the terminology used herein is for the purpose of describing particular embodiments only, and is not necessarily intended to be limiting. As used in this specification and the appended claims, the singular forms “a,” “an,” and “the” include plural referents unless the content clearly dictates otherwise. Thus, for example, reference to a “nucleic acid” optionally includes a combination of two or more nucleic acids, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology is used in accordance with the definitions set out herein.

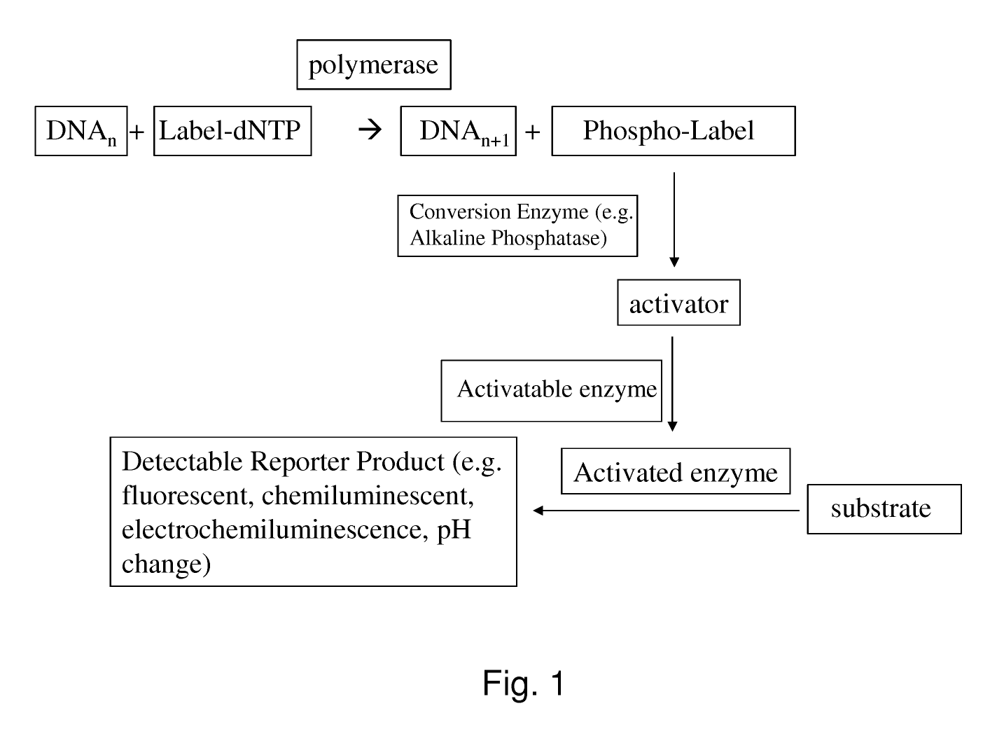

In general, the present invention provides reagents, methods, and systems/devices for efficient and low cost nucleic acid sequencing through monitoring of the generation of a phospho-product from nucleotide addition to a primed template. In various embodiments herein, a nucleotide (e.g. dATP, dTTP, dCTP, dGTP, ATP, UTP, CTP, GTP etc.) is modified to include a label attached through the terminal phosphate group. Such labels or phospho-labels can be an enzyme activator label, or a substrate which can be converted into an enzyme activator label. In the presence of a primed template and a polymerase activity, the labeled nucleotides can be added such that incorporation of a nucleotide by an enzyme such as a DNA polymerase to the primed template leads to generation of a phospho-product comprising the label. In particular embodiments, the phospho-product can be (or can comprise) an enzyme activator or can be converted to be or comprise an enzyme activator or, in the presence of a phosphatase, the phospho-product containing the label can be converted to an enzyme activator or to a substrate which can be converted to be or comprise an enzyme activator. In various embodiments, the label attached to the nucleotide can activate an activatable enzyme to a lesser extent than when the label is released from the nucleotide or modified after release, for example, dephosphorylation. In some embodiments, the enzyme activator increases the activity of the activatable enzyme which generates a detectable reporter. In some embodiments, the activated enzyme activity is used to generate a detectable signal, for example by fluorescence, luminescence or absorption but alternatively the signal can be detected by electrochemical detection, electrochemiluminescence, pH changes etc. A schematic illustration showing an exemplary embodiment of the invention is shown in FIG. 1. In the embodiments as shown in FIG. 1, once the phosphate is removed, the label is or can be converted (for example by a conversion enzyme) to an enzyme activator. In the presence of the inactive activatable enzyme, the phospho-label or a product of the label activates the enzyme which can directly or through a coupled reaction lead to generation of a detectable signal such as light or pH changes. In another embodiment, once the phosphate is removed, the dephosphorylated phospho-label can be a substrate for a conversion enzyme which generates an enzyme activator or multiple copies of an enzyme activator. For example, a nucleotide can be labeled with a maltodextrin. If a nucleotide is incorporated into a primed template, phospho-maltodextrin is released. In the prescence of a phosphatase and beta amylase, maltose is generated from the phospho-maltodextrin. The maltose can then act as an enzyme activator for a maltose activated enzyme. In another embodiment, the phospho-label can be converted to an enzyme activator.

In various embodiments, the process can be repeated, for example in a sequencing-by-synthesis scheme, to detect the incorporation of specific nucleotides and to obtain sequence information about the template. Individual templates or clonal copies of templates can be spatially separated so that sequencing can be performed on multiple templates in parallel. The methods herein can be used with multiple copies of clonal copies of nucleic acid templates or multiple copies of single molecule templates. The practice of the present invention can employ, unless otherwise indicated, conventional methods of chemistry, biochemistry, and recombinant nucleic acid techniques, within the skill of the art. Such techniques are explained fully in the literature and will be familiar to those of skill in the art.¹⁰ The reactions can optionally take place in microreactors, which microreactors optionally can be sealed, resealable or open.

In some embodiments, the nucleotides can be reversibly terminated such that after incorporation of a nucleotide, a reversibly coupled blocking group prevents addition of more nucleotides to the primed template. The release of the enzyme activator can be detected as described herein, e.g. above. The blocking group can then be removed, for example by light or a pH change, and more cycles of nucleotide addition conducted. This is particularly useful for accurately gauging the number of nucleotides in a homopolymer repeat region.

In some embodiments, nucleotides can have a different enzyme activator label, that is an enzyme activator that activates a different enzyme activity. For example, each nucleotide can have a distinct label. The labeled nucleotides can then be present simultaneously in a polymerization reaction. Incorporation of a nucleotide can be detected by detecting the particular enzyme activity which is activated. This can be optionally combined with the use of reversible terminators.

### DEFINITIONS

The following terms, as used herein, are intended to be defined as indicated below.

The singular terms “a”, “an,” and “the” as used herein include plural referents unless the content clearly dictates otherwise. Thus, for example, reference to “a nucleic acid” includes a mixture of two or more such nucleic acids, and the like.

The terms “polynucleotide,” “oligonucleotide,” “nucleic acid,” and “nucleic acid molecule” are used herein to include a polymeric form of nucleotides of any length, either ribonucleotides or deoxyribonucleotides or non-natural nucleotides such as protein nucleic acids. The nucleotides can be naturally occurring or synthetic. This term refers only to the primary structure of these molecules. Thus, the term includes triple-, double-, and single-stranded RNA and triple-, double-, and single-stranded DNA. It also includes modifications of these molecules, such as by methylation and/or by capping, and unmodified forms of the polynucleotide. There is no intended distinction in length between the terms “polynucleotide,” “oligonucleotide,” “nucleic acid,” and “nucleic acid molecule,” and these terms are used interchangeably herein. Thus, these terms include, for example, double- and single-stranded DNA, as well as double- and single-stranded RNA, DNA:RNA hybrids, and also include known types of modifications, for example, labels which are known in the art, methylation, “caps,” substitution of one or more of the naturally occurring nucleotides with an analog, as well as unmodified forms of the polynucleotide or oligonucleotide.

The terms “hybridize” and “hybridization” as used herein refer to the formation of complexes between nucleotide sequences that are sufficiently complementary to form complexes via Watson-Crick base pairing.

The term “particle” as used herein includes organic and inorganic beads (for example those made from glass, quartz or polymers), liposomes, highly branched polymers, quantum dots, and oil droplets.

The term “one copy” as used herein refers to a single molecule of a nucleic acid.

An “enzyme” as used herein refers to a macromolecule capable of catalyzing a biochemical reaction or a physical-chemical transformation. For example, enzymes can be proteins, protein complexes etc. which catalyze a reaction (e.g. proteases, lactamases, luciferases, etc.). As another example, an enzyme can be a ribozyme. In another example, an enzyme can be an ion channel which catalyzes the transfer of ions across a membrane barrier.

An “enzyme activator” herein comprises a compound which increases the activity of an enzyme. For example, allosteric activators increase the activity of an enzyme by binding to sites other than the active site of the enzyme. It will be appreciated that when enzyme activators as discussed herein, the methods of the invention also include those compounds that become enzyme activators once acted upon by a conversion enzyme.

A “nucleotide” is an organic molecule that serves as the monomers, or subunits, of nucleic acids like DNA and RNA. Nucleotides are composed of a nitrogenous base, a five-carbon sugar (ribose or deoxyribose), and at least one phosphate group. The sugar can be modified with a reversible termination group so that further polymerization requires removal of the reversible terminator.

### General

The present invention provides novel methods, systems and devices for sequencing nucleic acids efficiently and at very low cost. In various embodiments herein, a library containing a set of one or more copies of clonal nucleic acids is subjected to a sequencing reaction such as sequencing-by-synthesis. Sequential rounds of adding nucleotides that are labeled on the phosphate group can be conducted where the labeled dNTP or NTP is not an enzyme activator. Incorporation of a nucleotide and the number of incorporations per template can be determined by detection of the phosphoproduct of the polymerization reaction. If the phosphoproduct is not itself an enzyme activator, the phosphoproduct can be treated with one or more conversion enzyme (e.g., a phosphatase) to generate the enzyme activator or a substrate which can be used to generate the enzyme activator. In some embodiments the phospho-product itself is the enzyme activator or the substrate which can be used to generate the enzyme activator. The release of the label not attached via a phosphate to the nucleobase is indicative of incorporation of the nucleobase into the primed template. The release of the label can be detected via activation of an enzyme that is activated by the phospho-label or a product of the phospho-label where the enzyme can be detected in a coupled enzymatic reaction or by use of substrates which generate a detectable signal. For example, substrates can be used to generate signals such as: absorbance, fluorescence, luminescence, pH changes (which can be detected for example with ISFETs or pH sensitive fluorophores), conductivity, electrochemistry, currents from ligand gating ion channels, light from singlet oxygen beads (e.g. scintillation proximity assay), raman or electrochemiluminescence.

The methods herein can be used with single nucleic acid templates or with clonal copies of a template. The methods can be used with many ways of attaining clonal copies, for example by in vitro amplification of nucleic acids including emulsion PCR³ and bridge PCR⁵. The methods can also be used with rolonies³⁰. The templates can be attached to a surface (e.g. via streptavidin/avidin) or attached to particles which are attached to surfaces or placed in microwells which can be sealed or unsealed either when bound to a particle or to the microwell surface. In different embodiments, the method by which templates are bound to the particles can vary and, as with the various methods of attaining clonal copies, should not necessarily be taken as limiting. For example, target nucleic acids can be bound to particles by sequence specific capture oligos. In other examples, nucleic acids can be sheared, size fractionated, ligated with adaptors and captured by oligos on the particles which hybridize with the adaptors or non-covalently or covalently bound to the particles.

The number of copies of the target nucleic acid per site can also vary in the embodiments herein. For example, there can be one copy of the target sequence, up to 100 copies, up to 10,000 copies or more than 10,000 copies.

The choice of the label is also variable between the embodiments herein. For example, a label that can be used in various embodiments herein can comprise a beta-galactosidase donor peptide such as AcCGGGGXXXXXXGSLAVVLQRRDWENP GVTQLNRLAAHPPFASWRNSEEARTDCPSQQL circularized with a thiol crosslinker where XXXXXX represents a protease recognition sequence blocked by a phosphate or polyphosphate group. After release of the phospho-label following nucleotide incorporation, the phosphate or polyphosphate can be removed by a phosphatase. This allows the protease recognition site to be cleaved by a protease thereby linearizing the peptide. Once linearized, the peptide can be used to complement beta-galactosidase acceptor protein for generation of a detectable signal for example by using chromogenic, fluorogenic, protonogenic or luminescent substrates. As another example, in some embodiments, the label can be maltose which acts as an enzyme activator for the maltose switch protein developed by the Ostermeier group²¹. Allosteric enzyme activators increase the catalytic rate of an enzyme by binding to sites away from the active site and act to rapidly modulate enzyme catalyzed reactions in vivo²². The maltose switch protein system comprises a synthetic activated enzyme system which includes a fusion between maltose binding protein and a circularly permuted b-lactamase enzyme. The switch was designed for the in vivo detection of ligands. In the absence of maltose, maltose binding protein adopts an open conformation. Upon binding maltose, the protein hinges to close around the maltose. By incorporating a permuted b-lactamase within the maltose binding protein, the enzyme is activated upon closing of the hinge caused by maltose binding. The switch is specific for maltose, showing little activation by other sugars. The maltose-activated, b-lactamase switch protein can be used as the enzyme switch in the methods described herein. This switch protein has low enzymatic activity in the absence of maltose but activity increases several hundredfold (240 to 590) upon binding maltose. A variety of probes are available to detect b-lactamase activity including the chromogenic nitrocefin, fluorogenic fluorocillin, luminogenic Bluco²³ or protonogenic lactam antibiotics and can used with the various embodiments herein. By using this switch enzyme, the production of a single maltose molecule can lead to the activation of a single lactamase which then can generate many copies of a detectable reporter molecule. Generation of one or more maltose molecules can lead to generation of many fold more detectable reporter. Variants of the switch protein can also be used with the corresponding activator in the various embodiments herein. For example, the mutated protein which binds glucose can also be used. Alternatively, a histidine binding (or other periplasmic binding proteins or mutants of those proteins) can be substituted for the maltose binding protein in the switch. Other choices of activatable enzymes are also possible³¹ in other embodiments. For example, enzyme sensors which respond to ligand binding by increasing enzyme activity based on ubiquitin, b-galactosidase, dihydrofolate reductase (DHFR), b-lactamase, luciferase, inteins, barnase, proteases, glucokinase (GK), AMPactivated protein kinase (AMPK), p300 histone acetyltransferase, RNase L, and the sirtuin family of NAD+-dependent protein deacetylases have been reported³¹ and can be used in the various embodiments herein. In some embodiments, a ligand gated ion channel can be used by increasing channel conductance upon preferential binding of the phospho-label (i.e., after release of the phospho-label following nucleotide incorporation) or a conversion product of the phospho-label. Other activatable enzymes such as barnase, inteins, caspace, rybozymes, phosphatases, kinases, SIRT1 or lucifersases can also be used²⁴. In some embodiments, the signal-to-noise ratio for detection can be improved by increasing the activation ratio of the enzyme (i.e. the rate of enzyme activity in the presence of activator divided by the activity in the absence of activator). Activation ratios of 2, 4, 10, 100, 1,000, 10,000 or more are possible in various embodiments.

In some embodiments, as in the sequencing by synthesis approach used for the 454 pyrosequencing system⁴, solutions containing only one, two, or three of the four nucleotides (e.g., optionally labeled nucleotides) can optionally be added to the particles sequentially. Measurement of the enzyme activity after each reaction step can be used to determine whether one or more of a given nucleotide is incorporated. A DNA or RNA polymerase or reverse transcriptase can be used to incorporate the phospholabeled nucleotides.

The signal used to detect the presence of the enzyme activator can be varied. For example, the enzyme activator can be detected by fluorescence, chemiluminescence, absorbance, pH sensing (e.g. Ion Torrent system), electrochemiluminescence, coupled enzymatic reactions or by electrochemical methods. The reporter molecules (e.g. luminescent probes, protons, electrochemically active species etc.) can be detected using integrated CMOS sensors similar to that developed by Ion Torrent²⁵. For example, a CMOS ISFET sensor sensitive to pH or CMOS sensors capable of detection luminescence or even conductivity changes can be used.

The number of phosphates linking the label to the nucleotide can be varied to increase the efficiency of utilization of the labeled nucleotide by the polymerizing enzyme. For example, rather than 3 phosphates, 4 or 5 or more than 5 phosphates can be used.

In some aspects, the detection (e.g., optical detection or integrated CMOS sensor) is capable of measuring the label generated from an array of templates. The arrays can consist of 1, more than 1, more than 1,000, more than 1,000,000, or more than 1,000,000,000 templates. The array can be ordered or random. For example, arrays can consist of templates attached to a surface, attached to particles, templates within microwell arrays where the wells can be sealable or not sealable, etc. Examples of arrays include, but are not limited to, water in oil features, silicon microwells or selable microwells²⁶. The detection system used with the methods herein can be coupled to a computer, via an analog to digital or digital to analog converter, for transmitting detected light data to the computer for analysis, storage and data manipulation.

### Devices and Systems

Devices and systems that use the reagents and or practice the methods of the invention are a feature of the invention as well. The devices or systems can include an integrated reaction chamber and microreactor array (whether formatted as a consumable, or as dedicated portion of the device). Most typically, the devices comprise a signal detection subsystem, a fluidic module, a temperature or environmental control system and a computer with system instructions that control fluid exchange, detection, and post-signal processing, etc. The detection system can contain a fluorescence detection system, luminescence detection system or integrated CMOS sensors for detection of fluorescence, luminescence, absorbance, pH, conductivity, electrochemical reactions or the like. A temperature/environmental control module (e.g., comprising a Peltier device, cooling fans, etc.) can provide environmental control (e.g., temperature ramping). For fluorescence or absorbance detection, illumination light can be provided by one or more of a variety of sources (e.g., a lamp, arc lamp, LED, laser, or the like). An optical train can direct light from the illumination source to the microreactors. Signals from the microreactors can be detected by the detection system and unprocessed or partially processed signal information transmitted to a computer. The computer optionally also can control other system functions such as movement of reagents/solutions to and from microreactors, temperature control, etc. Signal information can be processed by the computer and outputted to a user viewable display, or to a printer, or to a storage device. A stage or mounting platform or holder is optionally present in some embodiments and can include registration and alignment features such as alignment arms, detents, holes, pegs, etc., that mate with corresponding features of the microreactors and/or other components. The devices can include a fluidic delivery system for delivering buffers and reagents to the microreactors. Fluid handling elements can be integrated into the devices or systems, or can be formatted into the microreactors. Fluid handling elements can include, but are not limited to, pipettors (manual or automated) that deliver reagents or buffers to ports in the consumable (e.g., to the microreactors), or can include capillaries, microfabricated device channels, or the like. The microreactor substrate optionally comprises ports that are configured to mate with the delivery system, e.g., ports of an appropriate dimension for loading by a pipette or capillary delivery device. The temperature/environmental control module can include features that facilitate thermocycling, such as a thermoelectric module, a Peltier device, a cooling fan, a heat sink, a metal plate configured to mate with one or more portions of an outer surface(s) of one or more of the components, a fluid bath, etc. Typically, such thermoregulatory component(s) have a feedback enabled control system operably coupled to a computer, which controls or is part of such. Computer directed feedback enabled control is an available approach to instrument control. In general, system control is performed by a computer, which can use, e.g., a script file as an input. If a nonintegrated detector is used for optical detection, the optical train can include any typical optical train components, or can be operably coupled to such components. The optical train directs illumination to the microreactors if needed. The optical train can also detect light (e.g., a fluorescent or luminescent signal) emitted from the microreactors. Typical optical train components can include any of an excitation light source, an arc lamp, a mercury arc lamp, an LED, a lens, an optical filter, a prism, a camera, a photodetector, a CMOS camera, and/or a CCD array. In one particular embodiment, an epifluorescent detection system is used. In certain aspects, the microreactors can be coupled directly to, or form part of, a detection system such as a CMOS sensor. Such sensors can be sensitive to light, pH, conductivity or other detectable signals. Alternatively, a fluidic interface, such as are present in conventional flow cytometers, can be provided on the detection channel in order to sample the polymerization reaction mixture. An optical detection system used for the invention will typically include one or more excitation light sources capable of delivering excitation light at one or more excitation wavelengths. Also included will be an optical train that is configured to collect the light emanating from the detection channel, and filter excitation light from the fluorescent signals. The optical train can also include additional separation elements for transmitting the fluorescent signals, and for separating the fluorescent signal component(s) emanating from the microreactors. The devices or systems can include or be operably coupled to system instructions, e.g., embodied in a computer or computer readable medium. The instructions can control any aspect of the devices or systems, e.g., to correlate one or more measurements of signal such as different signals detected due to incorporation of different nucleotides into a growing nucleic acid strand. A system can include a computer operably coupled to the other device components, e.g., through appropriate wiring, or through wireless connections. The computer can include instructions for normalizing signal intensity to account for background, e.g., for detecting local background for one or more regions of the microreactors, and for normalizing array signal intensity measurements by correcting for said background. In some embodiments, the microreactors can form part of a CMOS sensor. The reactors can be reversibly sealable, for example by pressing a gasket to the top of the microreactors. The CMOS sensor can function as the microreactor array, containing the sensing elements (for example pH, luminescence etc.) and can contain processing circuits to conduct some or all of the signal processing computations. In various embodiments, the CMOS sensor can be a disposable part of the device. The CMOS sensor also can be coupled to a flow cell to allow for exchange of reaction fluids. In some embodiments, the CMOS sensor can be temperature controlled.

Arrays of templates (either ordered or random) that can be used in the various embodiments herein can be made in microwells, on surfaces, within gels, within water in oil emulsion droplets, droplet arrays¹³ or other methods known in the art for separating templates. In some embodiments, it is preferred that the templates are enclosed within a diffusive barrier to prevent mixing of the products of multiple templates.

In the case of embodiments comprising fluorescent materials the detector can optionally include a light source that produces light at an appropriate wavelength for activating the fluorescent material, as well as optics for directing the light source through the detection window to the material contained in the sample cell. The light source can be any number of light sources that provides an appropriate wavelength, including lasers, laser diodes, and LEDs. Other light sources are used in other detection systems and can used with the various embodiments of the invention. For example, broad band light sources can be used in light scattering/transmissivity detection schemes, and the like.

### EXEMPLARY EMBODIMENTS OF THE INVENTION

Exemplary embodiments for carrying out the present disclosure are presented below. These embodiments are offered for illustrative purposes and are not intended to necessarily limit the scope of the invention in any way.

### Exemplary Embodiment 1

In some embodiments, clonal copies of templates can be confined near a surface. For example, individual molecules, rolonies or bead-attached clonal amplicons can be randomly bound to a surface, bound in ordered arrays on a surface, confined to microwells, confined in gells, or bound to hydrophilic spots on a hydrophobic surface to form femtoliter water in oil arrays. Labels can be used in some embodiments which require dephosphorylation and another conversion enzyme to convert the release of the phospho-label to the formation of the enzyme activator. The templates can be primed with an oligo. Buffers and reagents can be added to enable DNA polymerization and label detection. For example, in a suitable buffer (e.g. 10 mM Tris, 50 mM MgCl2, 5 mM MgCl2, pH 8) klenow exo-DNA polymerase, alkaline phosphatase, caspase 3, Fluorescein Di-β-D-Galactopyranoside and a beta-galactosidase enzyme acceptor can be used¹¹. To the templates can be sequentially added dNTPs (A, G, C and T) labeled with a circularized peptide such as:

AcCGGGGRVDEPFS(PO4)PGEKGSLAVVLQRRDWENPGVTQLNRLAAHP
PFASWRNSEEARTDCPSQQL
(circularized by C-C crosslinker)

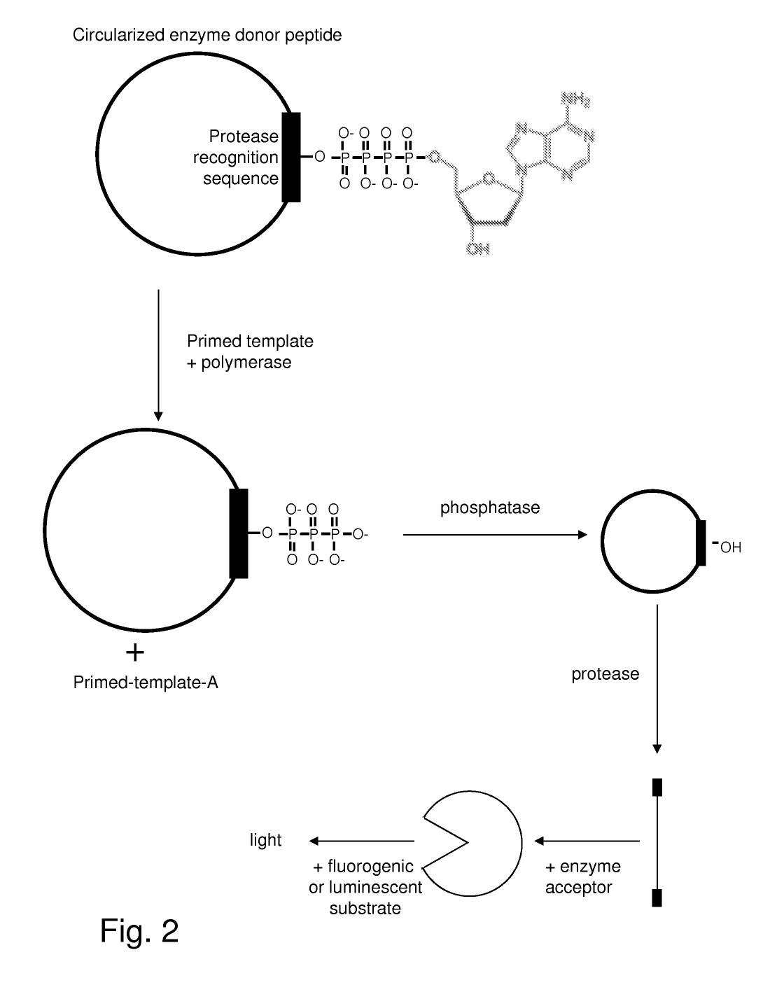

attached through the phosphoserine (see FIG. 2). Incorporation of a nucleotide can lead to generation of the phospho-circularized peptide which is dephosphorylated by the phophatase. The dephosphorylated peptide can be linearized by the caspase3. Once linearized, the donor peptide can complement the acceptor thereby generating an active beta galactosidase activity which forms fluorescein from the Fluorescein Di-β-D-Galactopyranoside substrate. The presence of the fluorecein can be measured by fluorescence microscopy after a suitable incubation time. The cycle can be repeated by washing away the reagents and adding another labeled dNTP.

### Exemplary Embodiment 2

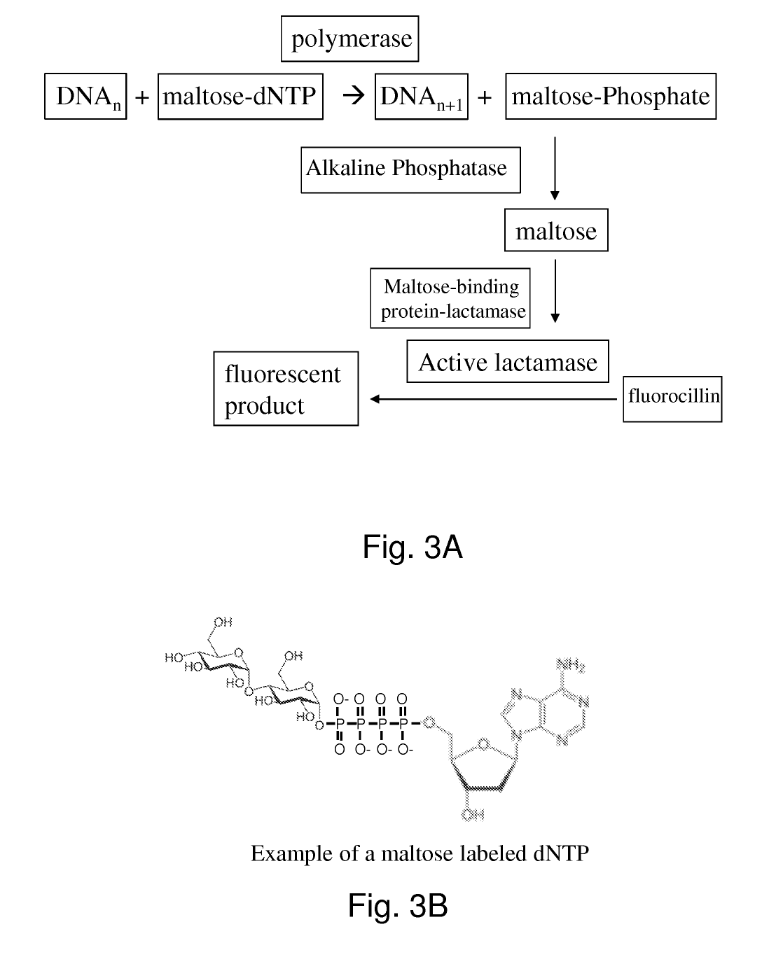

In some embodiments individual templates can be confined near a surface (e.g via capture of biotinylated templates on a streptavidin surface). For example, the templates can be randomly bound to a surface, bound in ordered arrays on a surface, confined to microwells, confined in gels, or bound to hydrophilic spots on a hydrophobic surface to form femtoliter water in oil arrays. The templates can be primed with an oligo or self primed. Buffers and reagents can be added to enable DNA polymerization and label detection. For example, in a suitable buffer (e.g. 10 mM Tris, 50 mM MgCl2, 5 mM MgCl2, pH 8) klenow exo-DNA polymerase, alkaline phosphatase, MBP-lactamase maltose sensor protein¹², and fluoricillin can be used (see FIG. 3). Maltase can be added at low activity to degrade any maltose formed before the beginning of the sequencing reaction. In some embodiments, a label can be used that requires dephosphorylation to generate an enzyme activator from the released phospho-label. To the templates can be sequentially added dNTPs (A, G, C and T) labeled with maltose attached through the phosphate. Incorporation of a nucleotide can lead to generation of the phospho-maltose which is dephosphorylated by the phophatase. Once dephosphorylated the maltose can bind to the MBP-lactamase and activate the lactamase. The increase in activity of the lactamase can be followed by the formation of a fluorescent product from the fluoricillin substrate. The presence of the product can be measured by fluorescence microscopy after a suitable incubation time. The cycle can be repeated by washing away the reagents and adding another labeled dNTP. The fluorescence signal for each cycle can be used to determine the polymer sequence.

### Exemplary Embodiment 3

In some embodiments tetraphosphate nucleotides labeled at the terminal phosphate with the enzyme activator maltose can be synthesized following the reaction scheme used to label nucleotides with fluorophores²⁸. Terminal phosphate labeled tetraphosphate nucleotides have been shown to be used by polymerases more efficiently than the corresponding triphosphates²⁸. Briefly, maltose-1-phosphate can be activated with carbonyldiimidazole and can be reacted in anhydrous DMF with the desired nucleotide to form a tetraphosphate nucleotide. The maltose-1-phosphate can be obtained by using maltokinase from Mycobacterium bovis BCG to phosphorylate maltose²⁹. The maltose-1-phosphate can be purified using anion exchange chromatography to reduce the maltose impurity. The product of the coupling reaction can be purified first by anion exchange chromatography, then by treatment with shrimp alkaline phosphatase to degrade any unreacted nucleotides followed by another round of anion exchange chromatography. The products can then be passed over a maltose binding protein column to further reduce any free maltose. Products can be characterized by LC/MS and phosphorous NMR to confirm the formation of the desired labeled tetraphosphate. Particularly for single molecule sequencing, it can optionally be important for the product to have minimal free maltose (<1 ppm) since the presence of even small quantities of free maltose can lead to false positive signals when doing single molecule sequencing. For femtoliter wells, 10 pM free maltose can result in ˜1% false positive rate for each nucleotide addition cycle. The maltose impurity level of the labeled dNTPs can be measured by monitoring spectrophotometrically the activation of the lactamase switch in the absence of phosphatase. Reagents can also be treated with low concentrations of maltase to degrade free maltose.

Bst 2.0 Warm Start polymerase (New England Biolabs, Ipswich, Mass.) can be used as the polymerase since Bst polymerase has been shown to efficiently incorporate terminal phosphate labeled nucleotides¹⁴. As described by Xie¹⁴, a temperature ramp can be used to start the polymerization. Using the Warm Start polymerase can allow for assembly of reactions at room temperature before activating the enzyme by warming the mix to >45° C.

The reaction flow cell and fluorescence detection system can be similar to that used by Xie¹⁴, Noji²⁶ and Walt²⁶. For multi-copy sequencing, 6 femtoliter silicon microwells (2 micron diameter) can be used. The microwell array can be housed within a flow cell made by sealing the array with double sided tape (3M, St. Paul, Minn.) to a PDMS gasket. One hole in the flow cell can serve as an inlet port for reagents while another hole can be attached to a vacuum line connected to a waste container. Pressure applied to the PDMS gasket can seal the wells after reagents have been added by pressure flow. The system can exchange buffers and seal the wells in less than 60 seconds. The flow cell can rest on a thermoelectric heater (VisionTek Systems, Cheshire, United Kingdom) to quickly bring the flow cell from room temperature to the ˜50° C. reaction temperature. Fluorescence detection can be performed on a Nikon Diaphot 300 microscope equipped with a 100-W mercury arc lamp, a 20×, 0.5 NA objective and a scientific SV643M CMOS camera (Epix, Buffalo Grove, Ill.). Images can be obtained at 1-2 Hz and the fluorescence intensity for individual wells summed using a custom MATLAB program (Mathworks, Natick, Mass.). The rate of change of fluorescence intensity can be calculated from the slope of the curve. The lactamase switch activity can be detected with the fluorogenic fluorocillin substrate.

Streptavidin beads, CP01N, 1.5 micron, (Bangs, Fishers, Ind.) can be labeled with 100 to 10,000 copies of short biotinylated primed oligonucleotide templates and loaded into the flow cell. At room temperature, reaction mix containing 5 uM nucleotide, Bst 2.0 WarmStart polymerase, shrimp alkaline phosphatase, 10 nM switch enzyme and 10 uM fluorocillin green can be flushed through the flow cell before sealing the PDMS gasket by applying vacuum to the waste well. The switch enzyme can be added quickly to the solution at 4° C., mixed and added to the wells. The temperature in the flow cell can then be ramped to >45° C. to initiate the polymerase reaction. Fluorescence can be monitored for 1-100 seconds. For multicopy sequencing, the detection reaction can be operated with the switch enzyme well below the Km in order to limit background fluorescence. Sufficient maltose can be generated to significantly activate the switch.

For single molecule sequencing, the background signal generated by unactivated lactamase switch can optionally be minimized by using very low volume wells. Silicon microwell arrays can be manufactured using standard photolithographic processes with cylindrical wells 0.5 to 1 um in diameter to give well volumes of 0.1 to 1 femtoliter. The micro-well slides can be placed in a flow cell developed and imaged as described above. The small volume can allow for micromolar reagent concentrations with only hundreds of reagent molecules per well. Instead of fluorocillin, any lactam such as cephalosporin can be used as a substrate to generate protons and detect the protons with the pH sensitive fluorescence detection of fluorescein. The lactamase switch can be immobilized on the surfaces of beads by using EDAC chemistry to couple anti-His tag antibody (Abcam, Cambridge, Mass.) to 0.5 to 1 um carboxy beads (Bangs) and attaching the lactamase switch via its His-tag. Biotinylated primed oligo templates can be attached to beads for sequencing. Beads can be reacted with both anti-His-tag antibody and avidin to provide binding sites for the lactamase switch and the template. The number of lactamase switches per bead can be varied by establishing a correlation between the concentration of the switch used during loading and the surface density of lactamase switch measured from the turnover of nitrocefin in a cuvette and comparing to a free lactamase switch calibration curve. After washing away uncaptured beads, Fluorocillin Green can be quickly flowed in, the wells sealed and the temperature ramped to reaction temperature. Primed templates can be used for sequencing with fluorescence detection following each round of dNTP introduction.

### Exemplary Embodiment 4

In another embodiment, activator sequencing can be used to sequence a nucleic acid without an instrument or with a reflection/absorbance/fluorescence scanner. A substrate, for example a hydrophobic membrane, can be treated to yield an array of small (0.5 to 50 micron) hydrophilic regions connected in series by hydrophilic strips. Each region can have an enzyme activator attached to the surface of the substrate and the activator can be chemically linked to the phosphate group of a dNTP. The linked dNTP can be patterned onto the regions in a desired order, for example (AGTC)ₙ. A clonal population of primed templates can be placed in the first region together with a reaction mix capable of polymerization and the material can move to successive regions by capillary action. If the primed template is in a region where a dNTP can be incorporated onto the primer, a dNTP can be incorporated and a phospho-activator can be left behind in the region. After the template has traveled from the first region to the final region, a signal can be developed by addition of phosphatase, the activatable enzyme and a substrate (chromogenic or fluorogenic) which forms a precipitating product. After sufficient time to develop a detectable signal, the substrate can be washed and analyzed. The pattern and intensity of regions containing precipitate can be indicative of the template sequence. The pattern and intensity can be detected visually, with a camera or a scanner.

### EXAMPLES

The following examples are offered to illustrate, but not necessarily to limit the claimed invention. One of skill will recognize a variety of non-critical parameters that may be altered without departing from the scope of the claimed invention. It is understood that the examples and embodiments described herein are for illustrative purposes only and that various modifications or changes in light thereof will be suggested to persons skilled in the art and are to be included within the spirit and purview of this application and scope of the appended claims.

Additionally, efforts have been made to ensure accuracy with respect to numbers used (e.g., amounts, temperatures, etc.), but allowances should be made for some experimental error and deviation.

### Example 1

A maltose with the 1′ hydroxyl attached to the terminal phosphate of a dNTP should not bind effectively to the switch and so should not activate the maltose binding protein/lactamase enzyme¹². The conjugated maltose/dNTP can be used to detect incorporation of dNTP during a cycle of sequencing by synthesis: the enzyme is only activated after incorporation of a dNTP released phospho-maltose which is then be dephosphorylated to yield the maltose activator. This scheme was demonstrated by synthesizing triphospho-maltose²⁷ and tested for its ability to activate the switch enzyme only upon dephosphorylation. Maltose monohydrate (1.44 g) was dissolved in 1 ml of water titrated to pH 12.5 by addition of NaOH and heated to 80° C. Trisodium trimetaphosphate (0.31 g) was added and the mixture reacted at room temperature for 25 hours. The maltose-triphosphate product was purified by anion exchange chromatography on a Dowex 1-X2 resin and eluted with 300 mM KCl. A fraction was isolated which showed a maltose to phosphate ratio of ˜3 (measured by microBCA and Biomol Green colorimetric assays after acid or phosphatase hydrolysis of the phosphates). The fraction showed a peak with mass 581 using LC/MS consistent with the formation of maltose-triphosphate.

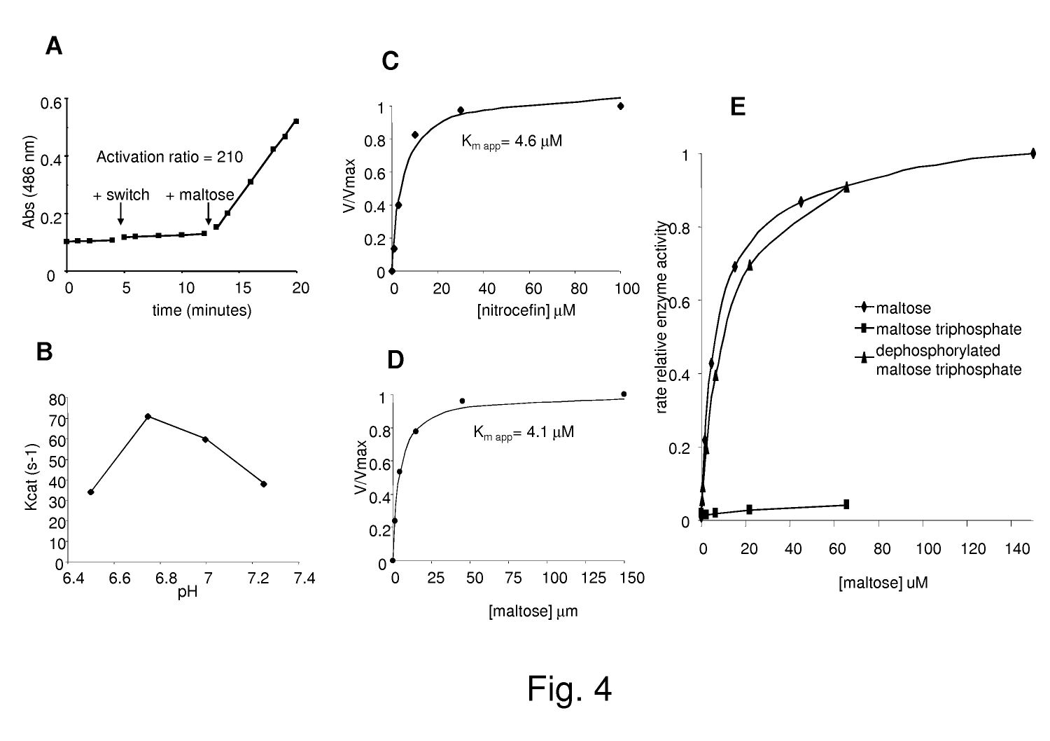

The switch protein was expressed and purified as previously described²¹ and enzyme activity was monitored using the chromogenic substrate nitrocefin. As seen in FIG. 4A, hydrolysis of nitrocefin was slow after addition of 10 nM switch enzyme but rapidly increased upon further addition of maltose. Addition of maltose increased the enzyme activity more than 210-fold. Kinetic analysis of the enzyme yielded a kcat value of 70 s-1 and apparent Km values of 4.6 and 4.1 mM for nitrocefin and maltose respectively (FIGS. 4B, 4C, and 4D). FIG. 4E shows that while maltose activates the protein, maltose triphosphate shows no appreciable activation (the small level of activity is consistent with the ˜2% impurity level of the maltose triphosphate). In contrast, addition of maltose triphosphate dephosphorylated with shrimp alkaline phosphatase (Affymetrix, Santa Clara, Calif.) showed activation levels similar to that of maltose. Taken together, these results demonstrate the ability of the switch enzyme in embodiments of the invention to differentiate between maltose and labeled maltose.

### Example 2

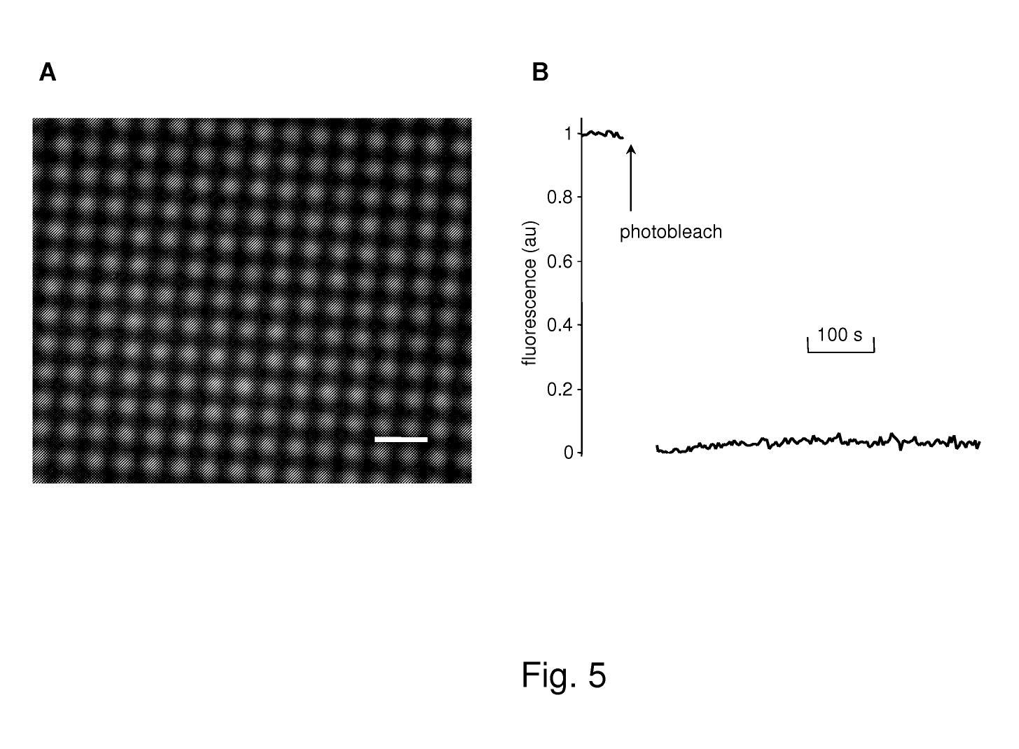

For a microwell with a volume of ˜1 fL, the single maltose molecule will be at a concentration well below the micromolar Km. However, several hundred switch proteins per microwell are sufficient to keep the enzyme concentration near the Km. Unfortunately, this leads to a high background from the residual activity of unactivated switch enzyme. This can be minimized by using an enzyme with a high activation ratio such as the lactamase switch and by using a very small well volume. Semiconductor microwells with submicron dimensions such as those used by the Ion Proton system²⁰ demonstrate the ability to generate billions of small wells. The microwells can be sealed with an 80 mm PDMS film attached to a 170 mm glass slide by applying a pressure of 25 psi and imaged using epifluorescence microscopy as shown in FIG. 5A. As shown in FIG. 5B, photobleaching of well fluorescence did not appreciably recover over hundreds of seconds indicating that the wells are well sealed. This shows the ability to seal a silicon well array to trap released activator during each sequencing-by-synthesis cycle.

While the foregoing invention has been described in some detail for purposes of clarity and understanding, it will be clear to one skilled in the art from a reading of this disclosure that various changes in form and detail can be made without departing from the true scope of the invention. All publications, patents, patent applications, and/or other documents cited in this application are incorporated by reference in their entirety for all purposes to the same extent as if each individual publication, patent, patent application, and/or other document were individually indicated to be incorporated by reference for all purposes.

### BIBLIOGRAPHY

¹Sanger F, Nicklen S, Coulson A R. DNA sequencing with chain-terminating inhibitors. Proc Natl Acad Sci USA. 1977 December; 74(12):5463-7.
²Smith L M, Sanders J Z, Kaiser R J, Hughes P, Dodd C, Connell C R, Heiner C, Kent S B, Hood L E. Fluorescence detection in automated DNA sequence analysis. Nature. 1986 Jun. 12-18; 321(6071):674-9. Rosenblum B B, Lee L G, Spurgeon S L, Khan S H, Menchen S M, Heiner C R, Chen S M. Nucleic Acids Res. 1997 Nov. 15; 25(22):4500-4. Links New dye-labeled terminators for improved DNA sequencing patterns.
³Dressman, D., Yan, H., Traverso, G., Kinzler, K. W. & Vogelstein, B. Transforming single DNA molecules into fluorescent magnetic particles for detection and enumeration of genetic variations. Proc. Natl. Acad. Sci. USA 100, 8817-8822 (2003). Tawfik D S, Griffiths A D. Man-made cell-like compartments for molecular evolution. Nat Biotechnol. 1998 July; 16(7):652-6.
⁴M. Margulies, et al. (2005). “Genome sequencing in microfabricated high-density picolitre reactors”. Nature 437: 376-380. J. Shendure, G. J. Porreca, N. B. Reppas, X. Lin, J. Pe McCutcheon, A. M. Rosenbaum, M. D. Wang, K. Zhang, R. D. Mitra and G. M. Church (2005). “Accurate Multiplex Polony Sequencing of an Evolved Bacterial Genome”. Science 309 (5741): 1728-1732.
⁵Adessi, C. et al. Solid phase DNA amplification: characterisation of primer attachment and amplification mechanisms. Nucleic Acids Res. 28, e87 (2000). Fedurco, M., Romieu, A., Williams, S., Lawrence, I. & Turcatti, G. BTA, a novel reagent for DNA attachment on glass and efficient generation of solid-phase amplified DNA colonies. Nucleic Acids Res. 34, e22 (2006)
⁶Fedurco, M., Romieu, A., Williams, S., Lawrence, I. & Turcatti, G. BTA, a novel reagent for DNA attachment on glass and efficient generation of solid-phase amplified DNA colonies. Nucleic Acids Res. 34, e22 (2006). Turcatti, G., Romieu, A., Fedurco, M. & Tairi, A. P. A new class of cleavable fluorescent nucleotides: synthesis and optimization as reversible terminators for DNA sequencing by synthesis. Nucleic Acids Res. 36, e25 (2008).
⁷Shendure, J. et al. Accurate multiplex polony sequencing of an evolved bacterial genome. Science 309, 1728-1732 (2005). McKernan, K., Blanchard, A., Kotler, L. & Costa, G. Reagents, methods, and libraries for bead-based sequencing. US patent application 20080003571 (2006).
⁸Quinn N L, Levenkova N, Chow W, Bouffard P, Boroevich K A, Knight J R, Jarvie T P, Lubieniecki K P, Desany B A, Koop B F, Harkins T T, Davidson W S. BMC Genomics. 2008 Aug. 28; 9:404. Assessing the feasibility of G S FLX Pyrosequencing for sequencing the Atlantic salmon genome.
⁹Palmer S, Kearney M, Maldarelli F, Halvas E K, Bixby C J, Bazmi H, Rock D, Falloon J, Davey R T Jr, Dewar R L, Metcalf J A, Hammer S, Mellors J W, Coffin J M. Multiple, linked human immunodeficiency virus type 1 drug resistance mutations in treatment-experienced patients are missed by standard genotype analysis. J Clin Microbiol. 2005 January; 43(1):406-13. Topaloglu O, Civin C I, Bunz F. Digital HLA allelotyping. Cancer Biol Ther. 2004 September; 3(9):899-902.
¹⁰See, e.g., A. L. Lehninger, Biochemistry (Worth Publishers, Inc., current addition); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); and Methods In Enzymology (S. Colowick and N. Kaplan eds., Academic Press, Inc.); Sambrook et al., Molecular Cloning—A Laboratory Manual (3rd Ed.), Vol. 1-3, Cold Spring Harbor Laboratory, Cold Spring Harbor, N. Y., 2000 (“Sambrook”).
¹¹U.S. Pat. No. 6,410,255 and Beta galactosidase enzyme fragment complementation as a high-throughput screening protease technology. Naqvi T, Lim A, Rouhani R, Singh R, Eglen R M. J Biomol Screen. 2004 August; 9(5):398-408.
¹²Guntas G, Mansell T J, Kim J R, Ostermeier M. Directed evolution of protein switches and their application to the creation of ligand-binding proteins. Proc Natl Acad Sci US A. 2005 Aug. 9; 102(32):11224-9.
¹³Sakakihara S, Araki S, lino R, Noji H. A single-molecule enzymatic assay in a directly accessible femtoliter droplet array. Lab Chip. 2010 Dec. 21; 10(24):3355-62.
¹⁴Sims P A, Greenleaf W J, Duan H, Xie X S. Fluorogenic DNA sequencing in PDMS microreactors. Nat Methods. 2011 Jun. 12; 8(7):575-80. Xie at al. US 2013/0053252 A1.
¹⁵Bentley, D. R., et al. “Accurate whole human genome sequencing using reversible terminator chemistry.” Nature, 2008, 456:53-9. Rothberg J M, et al. “An integrated semiconductor device enabling non-optical genome sequencing.” Nature, 2011, 475:348-52.
¹⁶Mashayekhi F and Ronaghi M. “Analysis of Read-Length Limiting Factors in Pyrosequencing Chemistry.” Anal Biochem., 2007, 363: 275-287.
¹⁷Eid, J., A. Fehr, J. Gray, K. Luong, J. Lyle, G. Otto, P. Peluso, et al. “Real-time DNA sequencing from single polymerase molecules.” Science, 2009, 323:133-138. Clarke, J., H. C. Wu, L. Jayasinghe, A. Patel, S. Reid, H. Bayley. “Continuous base identification for single-molecule nanopore DNA sequencing.” Nat. Nanotechnol., 2009, 4:265-70. Manrao E A, Derrington I M, Laszlo A H, Langford K W, Hopper M K, Gillgren N, Pavlenok M, Niederweis M, Gundlach J H. “Reading DNA at single-nucleotide resolution with a mutant MspA nanopore and phi29 DNA polymerase.”. Nat Biotechnol., 2012, 30:349-53. Cherf G M, Lieberman K R, Rashid H, Lam C E, Karplus K, Akeson M. “Automated forward and reverse ratcheting of DNA in a nanopore at 5-Å precision.” Nat Biotechnol., 2012, 30:344-8.
¹⁸Branton D, Deamer D W, Marziali A, Bayley H, Benner S A, Butler T, Di Ventra M, Garaj S, Hibbs A, Huang X, Jovanovich S B, Krstic P S, Lindsay S, Ling X S, Mastrangelo C H, Meller A, Oliver J S, Pershin Y V, Ramsey J M, Riehn R, Soni G V, Tabard-Cossa V, Wanunu M, Wiggin M, Schloss J A. “The potential and challenges of nanopore sequencing.” Nat Biotechnol., 2008, 26:1146-53. Peters B A, Kermani B G, Sparks A B, Alferov O, Hong P, Alexeev A, Jiang Y, Dahl F, Tang Y T, Haas J, Robasky K, Zaranek A W, Lee J H, Ball M P, Peterson J E, Perazich H, Yeung G, Liu J, Chen L, Kennemer M I, Pothuraju K, Konvicka K, Tsoupko-Sitnikov M, Pant K P, Ebert J C, Nilsen G B, Baccash J, Halpern A L, Church G M, Drmanac R. “Accurate whole-genome sequencing and haplotyping from 10 to 20 human cells.” Nature, 2012, 487:190-5.
¹⁹Eid, J., A. Fehr, J. Gray, K. Luong, J. Lyle, G. Otto, P. Peluso, et al. “Real-time DNA sequencing from single polymerase molecules.” Science, 2009, 323:133-138. Clarke, J., H. C. Wu, L. Jayasinghe, A. Patel, S. Reid, H. Bayley. “Continuous base identification for single-molecule nanopore DNA sequencing.” Nat. Nanotechnol., 2009, 4:265-70. Sims P A, Greenleaf W J, Duan H, Xie X S. “Fluorogenic DNA sequencing in PDMS microreactors.” Nat Methods, 2011, 8:575-80.
²⁰Merriman B; Ion Torrent R&D Team, Rothberg J M. “Progress in ion torrent semiconductor chip based sequencing.” Electrophoresis, 2012, 33:3397-417.
²¹Guntas G, Mansell T J, Kim J R, Ostermeier M. “Directed evolution of protein switches and their application to the creation of ligand-binding proteins.” Proc Natl Acad Sci USA, 2005, 102:11224-9.
²²Nussinov R, Tsai C J. “Allostery in disease and in drug discovery.” Cell, 2013, 153:293-305.
²³Yao H, So M K, Rao J. “A bioluminogenic substrate for in vivo imaging of beta-lactamase activity.” Angew Chem Int Ed Engl., 2007, 46:7031-4.
²⁴Hartley, Wood, Wells, Breaker, Chamberlin, Hardie, Westphal, Taneoka, Binkowski.
²⁵Yotter R A, Warren M R, Wilson D M. “Optimized CMOS photodetector structures for the detection of green luminescent probes in biological applications.” Sensors and Actuators B, 2004, 103:43-49. Esfandyarpour, H, Oldham M and Nordman E. “Methods and Systems for Electronic Sequencing.” US patent application 2013/0096013. Su X, Liu D. “Electronic Sensing for Nucleic Acid Sequencing.” U.S. Pat. No. 8,372,585. Pourmand N, Karhanek M, Persson H H, Webb C D, Lee T H, Zahradníková A, Davis R W. “Direct electrical detection of DNA synthesis.” Proc Natl Acad Sci USA, 2006, 103:6466-70.
²⁶Rondelez Y, Tresset G, Tabata K V, Arata H, Fujita H, Takeuchi S, Noji H. “Microfabricated arrays of femtoliter chambers allow single molecule enzymology.” Nat Biotechnol., 2005, 23:361-5. Gorris H H, Rissin D M, Walt D R. “Stochastic inhibitor release and binding from single-enzyme molecules.” Proc Natl Acad Sci USA, 2007, 104:17680-5. Merriman B; Ion Torrent R&D Team, Rothberg J M. “Progress in ion torrent semiconductor chip based sequencing.” Electrophoresis, 2012, 33:3397-417.
²⁷Inoue H, Tone N, Nakayama H, Tsuhako M. “Phosphorylation of disaccharides with inorganic cyclo-triphosphate in aqueous solution.” Chem Pharm Bull (Tokyo), 2002, 50:1453-9.
²⁸Sood A, Kumar S, Nampalli S, Nelson J R, Macklin J, Fuller C W. Terminal Phosphate-Labeled Nucleotides with Improved Substrate Properties for Homogeneous Nucleic Acid Assays. J. Am. Chem. Soc., 2005, 127:2394-2395.
²⁹Mendes V, Maranha A, Lamosa P, da Costa M S, Empadinhas N. “Biochemical characterization of the maltokinase from Mycobacterium bovis BCG.” BMC Biochem., 2010, 11:21.
³⁰Predki P F, Elkin C, Kapur H, Jett J, Lucas S, Glavina T, Hawkins T. Rolling circle amplification for sequencing templates. Methods Mol Biol. 2004; 255:189-96. Blab G A, Schmidt T, Nilsson M. Homogeneous detection of single rolling circle replication products. Anal Chem. 2004 Jan. 15; 76(2):495-8.
³¹Al-Horani R A, Liang A, Desai U R. Designing nonsaccharide, allosteric activators of antithrombin for accelerated inhibition of factor Xa. J Med Chem. 2011 Sep. 8; 54(17):6125-38. Vallee-Belisle A, Plaxco K W. Structure-switching biosensors: inspired by Nature. Curr Opin Struct Biol. 2010 August; 20(4):518-26. Stratton M M, Loh S N. Converting a protein into a switch for biosensing and functional regulation. Protein Sci. 2011 January; 20(1):19-29. Ostermeier M. Designing switchable enzymes. Curr Opin Struct Biol. 2009 August; 19(4):442-8. Skretas G, Wood D W. Regulation of protein activity with small-molecule-controlled inteins. Protein Sci. 2005 February; 14(2):523-32. Hartley R W. Complementation of peptides of barnase, extracellular ribonuclease of Bacillus amyloliquefaciens. J Biol Chem. 1977 May 25; 252(10):3252-4. Bishop A C, Chen V L. Brought to life: targeted activation of enzyme function with small molecules. J Chem Biol. 2009 March; 2(1):1-9.

What is claimed is:

1. A method for sequencing a nucleic acid, said method comprising the steps of:
a) immobilizing a target nucleic acid or a plurality of target nucleic acids in a microreactor;
b) introducing to the microreactor a mixture in solution phase, which mixture comprises a nucleic acid replicating catalyst and a first labeled nucleotide which first labeled nucleotide comprises a first base and a first label which first label does not substantially activate a first enzyme until after incorporation of said first labeled nucleotide into a complementary nucleic acid that is complementarity to said target nucleic acid;
c) performing template-dependent replication of said target nucleic acid or of the members of the plurality of target nucleic acids; and
d) detecting incorporation of said first labeled nucleotide during template-dependent replication by monitoring enzyme activity resulting from interaction of the first label with the first enzyme after release of said first label from the first labeled nucleotide, thereby sequencing said target nucleic acid.

2. The method of claim 1, wherein said mixture in solution phase further comprises a conversion enzyme that renders said first label capable of activating the first enzyme.

3. The method of claim 2, wherein said conversion enzyme comprises an alkaline phosphatase, acid phosphatase, galactosidase, horseradish peroxidase, phosphodiesterase, phosphotriesterase, pyruvate kinase, lactic dehydrogenase, maltose phosphorylase, glucose oxidase, lipase, protease, beta amylase, or any combination thereof of such enzymes.

4. The method of claim 1, wherein said first label is attached to the terminal phosphate of said first labeled nucleotide and wherein the label is cleavable from the first labeled nucleotide during replication of the nucleic acid complementary to the target nucleic acid.

5. The method of claim 1, wherein steps (b)-(d) are repeated with a second labeled nucleotide which nucleotide comprises a second base and a second label which second label does not substantially activate a second enzyme until after incorporation of said second labeled nucleotide into the complementary nucleic acid based on complementarity to said target nucleic acid, wherein the first and second labels are the same or different, wherein the first and second bases are different, and wherein the first and second enzymes are the same or different.

6. The method of claim 5, wherein steps (b)-(d) are repeated with a third labeled nucleotide which nucleotide comprises a third base and a third label which does not substantially activate a third enzyme until after incorporation of said third nucleotide into the complementary nucleic acid based on complementarity to said target nucleic acid, wherein any two of the first, second, and third labels are the same or different, wherein the first, second, and third bases are different, and wherein any of the first, second, and third enzymes are the same or different.

7. The method of claim 6, wherein steps (b)-(d) are repeated with a fourth nucleotide which nucleotide comprises a fourth base and a fourth label which does not substantially activate a fourth enzyme until after incorporation of said fourth nucleotide into the complementary nucleic acid based on complementarity to said target nucleic acid, wherein any two of the first, second, third, and fourth labels are the same or different, wherein the first, second, third, and fourth bases are different, and wherein any of the first, second, third, and fourth enzymes are the same or different.

8. The method of claim 7, further comprising sequentially repeating steps (b)-(d) with the first, second, third, and fourth nucleotides until the target nucleic acid is sequenced.

9. The method of claim 1, wherein the microreactor is reversibly sealed.

10. The method of claim 9, wherein exchange of components from the microreactor when it is sealed occurs through unsealing the reactor, removing the mixture in solution phase, introducing a second mixture in solution phase, and resealing the microreactor.

11. The method of claim 10, wherein the microreactor is sealed with a water-immiscible liquid or a PDMS gasket.

12. The method of claim 1, wherein said nucleic acid replicating catalyst comprises a DNA polymerase, an RNA polymerase, a ligase, a reverse transcriptase, or an RNA-dependent RNA polymerase.

13. The method of claim 1, wherein said target nucleic acid is DNA, and wherein said mixture in solution phase further comprises one or more nucleic acid primers.

14. (canceled)

15. The method of claim 1, wherein steps (b)-(d) are repeated to obtain the sequence for more than 1, more than 10, more than 25, more than 100, more than 300, more than 1,000 or more than 10,000 bases of said target nucleic acid.

16. The method of claim 1, wherein said target nucleic acid or plurality of target nucleic acids is immobilized on one or more beads disposed in said microreactor.

17. The method of claim 1, wherein said plurality of target nucleic acids is produced by rolling circle amplification.

18. The method of claim 1, wherein said target nucleic acid or plurality of target nucleic acids is immobilized on one or more surfaces of the microreactor.

19. (canceled)

20. The method of claim 1, wherein the first labeled nucleotide further comprises a reversible terminator.

21. The method of claim 20, further comprising wherein one or more of the second, third, or fourth labeled nucleotides comprises a reversible terminator.

22. (canceled)

23. (canceled)

24. (canceled)

25. The method of claim 1, further comprising more than one target nucleic acid or more than one plurality of target nucleic acids and wherein each target nucleic acid or each plurality of target nucleic acids is immobilized in one of a plurality of microreactors, and wherein steps (b)-(d) are performed for each target nucleic acid or each member of the plurality of target nucleic acids.

26. (canceled)

27. A method for sequencing a nucleic acid, the method comprising the steps of:
a) immobilizing a target nucleic acid or a plurality of target nucleic acids in a microreactor;
b) cooling said microreactor to 15° C. or lower;
c) introducing to the microreactor a mixture in solution phase which mixture comprises a nucleic acid replicating catalyst, and a first labeled nucleotide which labeled nucleotide comprises a first base and a first label which first label does not substantially activate a first enzyme until after incorporation of said nucleotide into a complementary nucleic acid that is complementary to said target nucleic acid;
d) sealing said microreactor and heating said microreactor to 30° C. or higher;
e) performing template-dependent replication of said target nucleic acid or of the members of the plurality of target nucleic acids;
f) detecting incorporation of said nucleotide during template-dependent replication by monitoring enzyme activity resulting from interaction of the label with the first enzyme after release of the first label from the first nucleotide, thereby sequencing the target nucleic acid; and,
g) repeating steps b)-f) with a second labeled nucleotide which second labeled nucleotide comprises a second base and a second label which second label does not substantially activate a second enzyme until after incorporation of said second nucleotide into the complementary nucleic acid; a third labeled nucleotide which comprises a third base and a third label which third label does not substantially activate a third enzyme until after incorporation of said third nucleotide into the complementary nucleic acid; and a fourth labeled nucleotide which comprises a fourth base and a fourth label which fourth label does not substantially activate a fourth enzyme until after incorporation of said fourth nucleotide into the complementary nucleic acid; wherein any of the first, second, third, and fourth labels are the same or different; wherein the first, second, third, and fourth bases are different; and wherein any of the first, second, third, and fourth enzymes are the same or different.

28. A system for sequencing a nucleic acid, the system comprising:
a) a plurality of microreactors that are each capable of holding: an immobilized target nucleic acid or plurality of target nucleic acids, a mixture in solution phase of a nucleic acid replicating catalyst, and one or more labeled nucleotides which labeled nucleotides each comprises a label that does not substantially activate an enzyme until after incorporation of said nucleotide into a complementary nucleic acid that is complementarity to said target nucleic acid;
b) a fluorescence or luminescence microscope or a luminescence or pH CMOS sensor for monitoring said plurality of microreactors by detecting in each microreactor the incorporation of an one or more labeled nucleotide into the complementary nucleic acid during or after template-dependent replication of said target nucleic acid by monitoring fluorescence, luminescence or pH changes resulting from cleaving of said labels from the labeled nucleotides when the nucleotides are incorporated into the complementary nucleic acid; and,
c) a fluidic delivery system capable of delivering liquids from one or more reservoirs to the members of said plurality of microreactors.

29. A compound comprising an enzyme activator or a label capable of being converted into an enzyme activator coupled to the terminal phosphate of a nucleotide.

30. A compound comprising the formula: 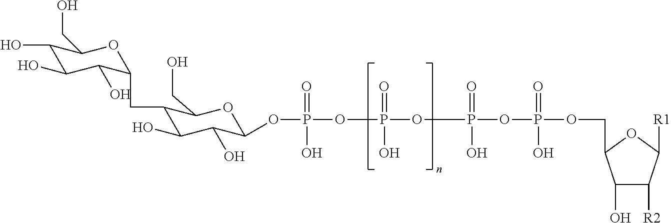
wherein n is 0 to 4, R1 is a nucleoside base and R2 is H, OH, or OMe.
